(19) United States
(12) Patent Application Publication
(10) Pub. No.: US 2004/0202704 A1
(43) Pub. Date: Oct. 14, 2004

(54) Transdermal delivery systems and methods

(75) Inventors: Sanjay Sharma, Sandy, UT (US); William Good, Salt Lake City, UT (US)

Correspondence Address: THORPE NORTH & WESTERN, P.O. BOX 1219, SANDY, UT, 84091-1219, US

(73) Assignee: Watson Pharmaceuticals, Inc.

(21) Appl. No.: 10/654,263

(22) Filed: Sep. 2, 2003

Related U.S. Application Data
(60) Provisional application No. 60/407,008, filed on Aug. 30, 2002.

(51) Int. Cl. A61K 9/70

(52) U.S. Cl. 424/449

(57) ABSTRACT

Transdermal systems for providing sustained delivery of a drug over a lengthy duration are disclosed and described. In certain aspects, such systems may take the form of a transdermal patch that includes, in addition to traditional components such as a backing member and a peelable release liner, a polymeric storage layer, a polymeric delivery layer, and a rate controlling member disposed therebetween.

### PRIORITY DATA

This application claims priority to U.S. Provisional Patent Application Serial No. 60/407,008, filed on Aug. 30, 2002, which is incorporated herein by reference.

### FIELD OF THE INVENTION

The present invention relates to transdermal delivery systems and method. Accordingly, the present invention involves the fields of pharmaceutical sciences, chemistry, medicine, and other health sciences.

### BACKGROUND

Transdermal drug delivery has become widely known as an alternative to traditional oral dosage therapy. Because of its nature and principles of operation, transdermal drug delivery provides a number of advantages for a patient over traditional oral dosage therapy, such as reduced administration frequency, and ease of administration. Such advantages generally minimize the inconvenience of dosage administration, and therefore improve patient compliance with a prescribed dosage regimen.

Additionally, transdermal drug delivery provides a number of pharmacokinetic advantages over traditional oral therapy. One example of such an advantage is the ability to achieve and maintain a steady state level of drug in the serum. The attainment of therapeutic steady state levels over a give duration optimizes the quality of therapy for the patient by stabilizing the effects of the drug and minimizing occasions where too much or too little drug is present.

An additional advantage of transdermal drug delivery is the ability to provide sustained delivery of a drug over a multi-day period. This improved delivery duration reduces the frequency of administration required, and thus improves patient compliance.

Fentanyl is one drug for which transdermal administration has been sought and commercialized. For example, the fentanyl patch disclosed in U.S. Pat. No. 4,588,580, which is incorporated herein by reference, is currently marketed under the trade name DURAGESIC™. See, Physicians Desk Reference, pg. 319, 1786 (56^{th} ed. 2002). Fentanyl is a strong analgesic which is used primarily to treat intense chronic pain, such as pain suffered by certain cancer patients. Because of the need for constant administration, and the desirability of sustained steady state blood levels, transdermal administration of fentanyl usually serves a patient well.

While many transdermal systems have been successful in providing sustained drug delivery for up to 3-5 days, few, if any have been able to realistically exceed this time period. In short, most transdermal systems are unable to hold enough drug to provide therapeutic steady state blood levels for such an extended administration, without becoming unstable. Further most transdermal systems fail to control the drug deliver at a rate that will provide such administration.

As such, transdermal delivery systems that can provide sustained drug delivery for a lengthy administration period continue to be sought through ongoing research and development efforts.

### SUMMARY OF THE INVENTION

Accordingly, the present invention provides a transdermal patch for administering a drug to a subject over an extended period of time, which includes:

a) a polymeric storage layer having an amount of the drug contained substantially therein, and having a proximal side and a distal side;

b) a polymeric delivery layer having an amount of the drug contained substantially therein, and having a proximal side and a distal side;

c) a backing member adhered to the distal side of the storage layer;

d) a rate controlling member adhered between the proximal side of the storage layer and the distal side of the delivery layer; and

e) a peelable release liner adhered to the proximal side of the delivery layer, wherein the release liner is removed prior to application of the patch to the skin of the subject. Such a transdermal patch has been found to affect sustained delivery of a drug for an administration period of greater than three days. In some aspects, the duration of administration may be about a 7-10 days or more.

A number of the materials may be used for the polymeric storage layer, and the polymeric delivery layers used in the transdermal delivery devices of the present invention. Examples of such polymeric materials may include, without limitation, acrylate polymers, vinyl polymers, silicone polymers, polyisobutylene polymers, copolymers thereof, and mixtures thereof. However, in one aspect, the polymeric material of the storage layer may be a polyvinyl pyrrolidone polymer or copolymer, such as a polyvinyl pyrrolidone acrylate. In another aspect, the polymeric material of the delivery layer may be a silicone polymer.

The rate controlling member used in the transdermal devices of the present invention may be made from a wide variety of materials and structures known to those of ordinary skill in the art for controlling the rate at which a drug is released from a carrier. However, in one aspect, the rate controlling member may be a structure, such as a microporous membrane, or dense material. In another aspect, the rate controlling member a may be a layer of a polymeric material. In an additional aspect, the rate controlling member may be a layer that includes ethylene vinyl acetate (EVA).

In order to provide a sustained delivery, the amount or concentration of drug initially present in both of the storage and delivery layers may be specifically selected. Varying amounts may be selected by one of ordinary skill in the art in order to obtain a device that is suitable for administration over a selected period of time. Moreover, the amount of drug contained in either the storage or delivery layer may be adjusted in proportion to the other layer in order to affect a specific delivery profile, or duration of administration. However, in one aspect, the amount of drug initially present in the storage layer may be from about 4 to about 15 times the amount of drug in the delivery layer. In another aspect, the amount of drug initially present in the storage layer may be about 5 times the amount of drug in the delivery layer.

The amount and type of drug used in the transdermal devices of the present invention may be selected by one of ordinary skill in the art, in order to provide a medication suitable to treat a specific illness. A list of exemplary drugs is recited below. However, in one aspect, the drug may be fentanyl. While the fentanyl concentration provided in each layer may be selected as required in order to provide a specific release profile or administration period, in one aspect, the amount of fentanyl in the storage layer may be from about 2% w/w to about 12% w/w of the storage layer, and the amount of fentanyl in the delivery layer it is from about 0.5% w/w to about 1.2% w/w of the delivery layer. In yet another aspect, the amount of fentanyl in the storage layer is about 8% w/w of the storage layer, and the amount of fentanyl in the delivery layer is about 0.8% w/w of the delivery layer.

In addition to the transdermal delivery devices enumerated herein, the present of the invention further encompasses a method for providing extended transdermal delivery of a drug to a subject. In one aspect, such a method may include administering a transdermal patch as recited herein to the skin of the subject.

There has thus been outlined, rather broadly, the more important features of the invention so that the detailed description thereof that follows may be better understood, and so that the present contribution to the art may be better appreciated. Other features of the present invention will become clearer from the following detailed description of the invention and claims, or may be learned by the practice of the invention.

### BRIEF DESCRIPTION OF DRAWINGS

FIG. 1 is a graphical representation of fentanyl delivery information obtained using three different embodiments of transdermal delivery systems in accordance with the present invention.

FIG. 2 is a graphical representation of fentanyl delivery information obtained using three different embodiments of transdermal delivery systems in accordance with the present invention as compared to a monolithic fentanyl containing adhesive matrix patch marketed by under the trade name DURAGESIC™.

### DETAILED DESCRIPTION OF THE INVENTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “an adhesive” includes reference to one or more of such adhesives, and reference to “the excipient” includes reference to one or more of such excipients.

As used herein, “subject” refers to a mammal that may benefit from the administration of a drug composition or method of this invention. Examples of subjects include humans, especially females, and may also include other animals such as horses, pigs, cattle, dogs, cats, rabbits, and aquatic mammals.

As used herein, the terms “formulation” and “composition” are used interchangeably. The terms “drug,” “pharmaceutical,” “active agent,” and “bioactive agent” are also used interchangeably to refer to a pharmacologically active substance or composition. These terms of art are well-known in the pharmaceutical and medicinal arts.

As used herein, the terms “administration,” and “administering” refer to the manner in which a drug is presented to a subject. Administration can be accomplished by various routes well-known in the art such as oral, and non-oral methods.

As used herein, “transdermal” refers to the route of administration that facilitates transfer of a drug through a skin surface wherein a transdermal composition is administered to the skin surface.

As used herein, “skin,” “skin surface,” “derma,” and “epidermis” may be used interchangeably, and are meant to include not only the outer skin of a subject comprising one or more of epidermal layers, but also to include mucosal surfaces to which a drug composition may be administered. Examples of mucosal surfaces include the mucosa of the respiratory (including nasal and pulmonary), oral (mouth and buccal), vaginal, and rectal cavities. Hence the terms “transdermal” may encompass “transmucosal” as well.

As used herein, “enhancement,” “penetration enhancement,” or “permeation enhancement,” refer to an increase in the permeability of the skin, to a drug, so as to increase the rate at which the drug permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of permeation enhancers is disclosed by David W. Osborne and Jill J. Henke, in their publication entitled Skin Penetration Enhancers Cited in the Technical Literature, published in “Pharmaceutical Technology” (June 1998), which may also be found at the worldwide web address known as: pharmtech.com/technical/osborne/osborne.htm, which is incorporated by reference herein.

An “effective amount” of an enhancer refers to an amount sufficient to increase penetration of a drug through the skin, to a selected degree. Methods for assaying the characteristics of permeation enhancers are well-known in the art. See, for example, Merritt et al., Diffusion Apparatus for Skin Penetration, J. of Controlled Release 61 (1984), incorporated herein by reference in its entirety. By “effective amount” or “therapeutically effective amount,” or similar terms is meant a non-toxic but sufficient amount of a drug, to achieve therapeutic results in treating a condition for which the drug is known to be effective. The determination of an effective amount is well-within the ordinary skill in the art of pharmaceutical and medical sciences. See for example, Curtis L. Meinert & Susan Tonascia, Clinical Trials: Design, Conduct, and Analysis, Monographs in Epidemiology and Biostatistics, vol. 8 (1986).

As used herein, “pharmaceutically acceptable carrier,” and “carrier” may be used interchangeably, and refer to any inert and pharmaceutically acceptable material that has substantially no biological activity, and makes up a substantial p art of the formulation. The carrier may be polymeric, such as an adhesive, or non-polymeric and is admixed with other components of the composition (e.g., drug, binders, fillers, penetration enhancers, anti-irritants, emollients, lubricants, etc., as needed) to comprise the formulation.

The term “admixed” means that the drug and/or enhancer can be dissolved, dispersed, or suspended in the carrier.

As used herein the term “matrix”, “matrix system”, or “matrix patch” is meant a composition comprising an effective amount of a drug dissolved or dispersed in a polymeric phase, which may also contain other ingredients, such as a permeation enhancer diluents, skin irritation reducing agents, excipients, plasticizers, emollients, and other optional ingredients. This definition is meant to include embodiments wherein such polymeric phase is laminated to a pressure sensitive adhesive or used within an overlay adhesive.

A matrix system may also comprise an adhesive layer having an impermeable film backing attached onto the distal surface thereof and, before transdermal application, a release liner on the proximal surface of the adhesive. The film backing protects the polymeric phase of the matrix patch and prevents release of the drug and/or optional ingredients to the environment. The release liner functions similarly to the impermeable backing, but is removed from the matrix patch prior to application of the patch to the skin as defined above. Matrix patches with the above-described general characteristics are known in the art of transdermal delivery. See, for example, U.S. Pat. Nos. 4,588,580, 5,985,317, 5,783,208, 5,626,866, 5,227,169, which are incorporated by reference.

As used herein, “delivery layer”, refers to a layer of a polymeric material that has a propensity to dispense a drug therefrom. In some cases, such a propensity may be due to the fact that the material has a capacity to dissolve and store the drug which is lower than the capacity of a “storage layer” used in the same transdermal device for dissolving and storing the same drug. As such, a “delivery layer” may in some aspects, have a solubility capacity for a given drug, that is lower than the solubility capacity of a “storage layer” for that same drug.

As used herein, “storage layer”, refers to a layer of a polymeric material that has a greater capacity for dissolving and storing a drug than the capacity of a delivery layer used in the same transdermal device for dissolving and storing the same drug. As such, a “storage layer” may in some aspects, have a solubility capacity for a given drug that is higher than the solubility capacity of a “delivery layer” for that same drug.

As used herein, “fentanyl” refers to a compound having the general structure:

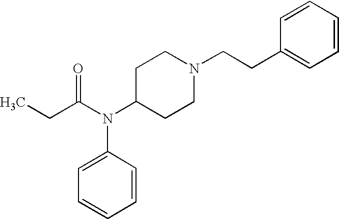

Fentanyl is a well known analgesic, and is included as entry no. 4043 on pg. 679-680 of the Merck Index 12^{th} ed. (1996), which is incorporated herein by reference. Further, as used herein, “fentanyl” is meant to include, prodrugs, salts, metabolites, and other related compounds thereof.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a range of 0.1 to 5 should be interpreted to include not only the explicitly recited concentration limits of 0.1 and 5, but also to include individual concentrations such as 0.2, 0.7, 1.0, 2.2, 3.6, 4.2, and sub-ranges such as 0.3-2.5, 1.8-3.2, 2.6-4.9, etc. This interpretation should apply regardless of the breadth of the range or the characteristic being described and should also apply to open ended ranges, for example, “less than 5” or “greater than 10”.

The Invention

As recited above, the present invention provides transdermal systems and methods for effecting extended drug delivery. In certain aspects, a drug may be administered on a substantially continuous basis for a duration of more than about 3 days. In other aspects, the duration of administration may be from about 7 to about 10 days, or longer.

One mechanism by which extended drug administration may be achieved, is with the use of a multilayer transdermal patch. In one aspect, such a patch may include:

a) a storage layer having an amount of the drug contain substantially therein, and having a proximal side and a distal side;

b) a delivery layer having an amount of the drug contain substantially therein, and having a proximal side and a distal side;

c) a backing member adhered to the distal side of the storage layer;

d) a rate controlling member adhered between the proximal side of the storage layer and the distal side of the delivery layer; and

e) a peelable release liner adhered to the proximal side of the delivery layer. Obviously, in using such a device, the release liner is removed prior to application of the device to the skin.

The storage layer utilized in the device of the present invention, may be a polymeric material that has a high capacity for dissolving and storing the drug to be delivered. Additionally, the delivery layer utilized in the device of the present may be invention may be a polymeric material having a lower capacity for dissolving or storing the drug than that of the storage layer. In some aspects, this lower capacity may contribute to the predisposition of the delivery layer to dispense the drug to the skin in a quick, or timely fashion. When separated by a rate controlling membrane, a solubility differential is created between the layers that may aid in the ability to sustain or extend drug administration to the skin.

In practice, drug contained in the delivery layer is dispensed to the skin, and drug contained in the storage layer is then dispensed into the delivery layer at a metered rate through the rate controlling member. By employing this arrangement, it has been discovered that a greater amount of total drug can be loaded into the transdermal device as compared to monolithic transdermal devices. Both the total amount of drug contained in the patch, as well as the differential solubility created by the multilayer design may play a role in achieving extended drug delivery. Strong substantially constant delivery is important to the attainment of steady state serum levels over an extended duration.

The specific type of drug utilized in the devices of the present invention, and the amount of such drug in each layer may be a matter of choice by one of ordinary skill in the art according to the intended medicament to be formed. Examples of drug types useful in the present invention are elaborated more fully below. In one aspect, the amount of drug in each layer may be determined by the amount of drug to be included in the other layer. For example, in one aspect, the amount of drug initially present in the storage layer may be from about 4 to about 15 times greater than the amount of drug initially present in the delivery layer. In another aspect, the amount of drug initially present in the storage layer may be about 5 times greater than the amount of drug initially present in the delivery layer.

A wide range of polymeric materials, including adhesives, may be used either of the storage layer, or delivery layer utilized by the transdermal delivery systems of the present invention. The specific selection of a particular polymeric material for a given layer may be made by one of ordinary skill in the art based on a number of factors, such as compatibility with the agents or components to be combined therewith, and its interaction with supporting structures, such as the backing member and release liner. However, the primary factor in selection of a particular polymeric material will typically be the capacity of the material to receive and hold a given drug, or its propensity to dispense or release a given drug. As noted above, those polymeric materials selected for the storage layer will generally have a high capacity for receiving a given drug, which will enable the incorporation of a high concentration of drug thereinto. By contrast, those polymeric materials selected for the delivery layer will generally have a propensity to quickly, or timely release or dispense the drug therefrom. Most often the polymeric materials selected for the storage layer and the delivery layer will be different, the selection of which will be determined by the interactive nature of the material with the drug to be delivered.

In one aspect of the polymeric materials for either the storage layer or the delivery layer may include acrylate polymeric materials, silicon polymeric materials, and rubber-based polymeric materials.

Examples of acrylic polymeric materials, such as acrylate polymers include without limitation any of the homopolymers, copolymers, terpolymers, and the like of various acrylic acids. In another aspect of the invention, the acrylate polymers may be a combination of one or more monomers of acrylic acids and other copolymerizable monomers.

Acrylate polymers may also include copolymers of alkyl acrylates and/or methacrylates, and/or copolymerizable secondary monomers or monomers with functional groups. Specific examples of acrylate monomers, which are suitable for use with the present invention include, but are not limited to methacrylic acid, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecylmethacrylate, tridecyl acrylate, tridecyl methacrylate, and mixtures thereof.

Specific examples of functional monomers which are copolymerizable with the above-recited alkyl acrylates or methacrylates, which can also be used include, but are not limited to acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxethyl acrylate, methoxyethyl methacrylate, and mixtures thereof.

Further details and examples of acrylic adhesives which are suitable for use in the present invention are set forth in Satas, “The Handbook of Pressure-sensitive Adhesive Technology,” 2^{nd} ed, Pp. 396-456 (1989), which is incorporated herein by reference in its entirety.

Examples of suitable acrylic adhesives which are commercially available include polyacrylate adhesives sold under the trademarks DUROTAK® by National Starch and Chemical Corporation, Bridgewater, N.J., as well as GELVA-MULTIPOLYMER SOLUTION® Monsanto, St. Louis, Mo. Other examples of adhesives, and adhesive formulations, which can be used in connection with the present invention are disclosed in U.S. Pat. No. 5,656,286, which is incorporated herein by reference in its entirety.

In one aspect, the polymeric material of the storage layer may be an acrylic copolymer of polyvinyl pyrrolidone, such as polyvinyl pyrrolidone acrylate.

Specific examples of rubber-based pressure sensitive adhesives include, but are not limited to hydrocarbon polymers, such as natural and synthetic polyisoprenes, polybutylenes and polyisobutylene (PIB), styrene/butadiene polymers, styrene-isoprene-styrene block copolymers, hydrocarbon polymers such as butyl rubber, halogen-containing polymers such as polyacrylic nitrile, polytetrafluoroethylene, polyvinyl chloride, polyvinylidene chloride, and polychlorodiene, and other copolymers thereof. Examples of PIB adhesives may be found in U.S. Pat. No. 5,508,308, which is incorporated herein by reference.

Specific examples of silicone polymers, or polysiloxanes include but are not limited to silicone pressure sensitive adhesives, which are a based on two major components: a polymer, or gum, and a tackifying resin. The polysiloxane adhesive may be prepared by cross-linking the gum, typically a high molecular weight polydiorganosiloxane with the resin to produce a three-dimensional silicate structure via a condensation reaction in an appropriate organic solvent. Various aspects of formulating polysiloxane adhesives are disclosed by Sobieski et al, in “Silicone Pressure sensitive Adhesives,” I.d. at pg. 508-517. Silicone pressure-sensitive adhesives are commercially available and include the silicone adhesives sold under the trademarks BIO-PSA® Dow Corning Corporation, Medical Products, Midland, Mich.

In one aspect, the polymeric material of the delivery layer may be a silicone polymer, or silicone adhesive.

A rate controlling member is included as part of the transdermal delivery device of the present invention. Generally, the rate controlling member is adhered between a proximal side of the storage layer, and a distal side of the delivery layer. The rate controlling member is provided for the purpose of metering, or controlling, the rate at which drug migrates from the storage layer into the delivery layer.

Those of ordinary skill in the art will recognize a number of specific materials and structures, which may be used as the rate controlling member of the present invention. For example, the rate controlling member may be a “dense” membrane made of a material that is inherently permeable to the drug and other components of the storage layer, which are to be conveyed to the delivery layer. Alternatively, it may be made of a microporous material whose pores are filled with a drug-permeable material. Further, the rate controlling member may be a layer made of a specific polymeric material. For example, in one aspect, the rate controlling member may include an amount of ethylene vinyl acetate (EVA) copolymer. In another aspect, the amount of EVA contained in the rate controlling member may be from about 1% w/w to about 20% w/w of the rate controlling member. In yet another aspect, the amount may be from about 2% w/w to about 9% w/w of the rate controlling member.

In the case of dense membranes, the component(s) traveling from the storage layer to the delivery layer typically dissolve in the material and diffuse through to the delivery layer. In the case of microporous materials the component(s) diffuse through the pores to the delivery layer. Examples of materials for making dense membranes are given in U.S. Pat. Nos. 3,598,122 and 4,650,484. Examples of materials for making microporous membranes are provided in U.S. Pat. Nos. 3,797,494 and 4,031,894.

Those of ordinary skill in the art will recognize that the transdermal patch of the present invention may be specifically constructed, or adapted in order to accommodate extended delivery of a wide number of specific drugs. Examples of suitable drugs generally include therapeutic agents in all of the therapeutic areas including, but not limited to: antibiotics (including antimicrobials, antibacterials, antimycobacterials, antimalerials, antiamebics, anthelminics, antifungals, and antivirals), neoplastic agents, agents affecting the immune response (including steroidal and non-steroidal anti-inflammatory agents), blood calcium regulators, peptide and protein hormones, agents useful in glucose regulation, antithrombotics and hemostatics, antihyperlipidemic agents, thyromimetic and antithyroid drugs, antiulcer agents, histamine receptor agonists and antagonists, inhibitors of allergic response, local anesthetics, analgesics and analgesic combinations, antipsychotics, anti-anxiety agents, antidepressants agents, anorexigenics, bone-active agents, diagnostic agents, and a mixture thereof. Additional examples include: antidiarrheals, antimigraine preparations, antimotion sickness agents, antinauseants, antiparkinsonism drugs, antipruritics, antipyretics, antispasmodics (including gastrointestinal, urinary, skeletal, and smooth-muscle), anticholinergics, sympathomimetics, xanthine derivatives, cardiovascular preparations (including calcium channel blockers, beta-blockers, antiarrythmics, antihypertensives, diuretics, vasodilators including general coronary, peripheral and cerebral), central nervous system stimulants including cough and cold preparations, decongestants, diagnostics, hormones, immunosuppressives, parasympatholytics, parasympathomimetics, sedatives, tranquilizers and mixtures thereof.

Examples of specific drugs include without limitation: antibiotics, such as amoxicillin, cloxacillin sodium, penicillin G potassium; antimicrobials: benzalkonium chloride, chlorohexidine, gluconate hexachlorophene; antibacterials, such as sulfabenzamide, sulfadiazine, sulfasalazine; antimycobacterials, such as chlofazimine, ethambutol, isoniazid; antimalerials, such as chloroquine hydrochloride, quinine sulfate, pyrimethamine; antiamebics, such as arsthinol, bialamicol, carbarsone; anthelminics, such as ivermectin, bithionol, piperazine; antifungals, such as clotrimazole, griseofulvin, miconazole; antivirals, such as acyclovir, foscarnet sodium, ribavirin; neoplastic agents, such as adriamycine, cyclophosphamide, methotrexate; immune response steroidal anti-inflammatory agents, such as hydrocortisone, dioxyanthranol, betamethasone; non-steroidal anti-inflammatory agents (NSAIDs), such as choline salicylate, diflunisal, ibuprofen, acetaminophen; blood calcium regulators, such as parathyroid hormone, calcifediol, calcitonin; peptide and protein hormones, such as insulin, glucagon, vasopressin; glucose regulators, such as tolazamide, tolbutamide, chlorpropamide; antithrombotics, such as aspirin, sulfinpyrazone, dipyridamole; hemostatics, such as thrombin, microfibrillar collagen, absorbable gelatin powder; antihyperlipidemic a gents, such as pravastatin sodium, simvastatin, clinofibrate; thyromimetic and antithyroid drugs, such as methimazole, propylthiouracil, potassium iodide; antiulcer agents, such as metoclopramide, histidine hydrochloride, famotidine; histamine receptor agonists and antagonists, such as astemizole, clemastine fumarate, cyclizine; allergic response inhibitors, such as astemizole, clemastine fumerate, diphenhydramine hydrochloride; local anesthetics, such as chloroprocaine hydrochloride, lidocaine hydrochloride, procaine hydrochloride; analgesics and analgesic combinations, such as acetaminophen, aspirin, ibuprofen; antipsychotics, such as acetophenazine maleate, chlorprotbixene, droperidol; anti-anxiety agents, such as diphenhydramine, phenobarbital, chlordiazepoxide; anti-depressants, such as amitriptyline hydrochloride, amoxapine, fluoxetine hydrochloride; anorexigenics, such as amphetamine, methamphetamine, chlorphentermine; bone-active agents, such as parathyroid hormone, calcitonin; diagnostic agents, such as benzylpeniclloyl polylysine, iocetamic acid, aminohippurate sodium; antidiarrheals, such as diphenoxylate hydrochloride, loperamide hydrochloride, fennel oil; antimigraine preparations, such as dihydroergotamine mesylate, ergotamine tartrate, methysergide maleate, sumatriptin succinate; antimotion sickness agents, such as buclizine hydrochloride, diphenidol, meclizine hydrochloride; antinauseants, such as benzquinamide hydrochloride, dronabinol, dimenhydrinate; antiparkinsonism drugs, such as amantadine hydrochloride, benztropine mesylate, biperiden hydrochloride; antipruitics, such as camphor, menthol, pramoxine; antipyretics, such as acetaminophen, aspirin, ibuprofen; antispasmodics (including gastrointestinal, urinary, skeletal and smooth-muscle), such as flavoxate, flavoxate hydrochloride, ethaverine hydrochloride, oxybutynin chloride, dicyclomine; anti-cholinergics, such as propantheline, oxybutynin, oxybutynin hydrochloride, adiphenine hydrochloride, aminopentamide, atropine; sympathomimetics, such as dopamine hydrochloride, epinephrine, ephedrine sulfate; xanthinederivatives, such as caffeine, theophylline, aminophylline; calcium channel blockers, such as amlodipine, felodipine, isradipine, diltiazem, nifedipine; beta blockers, such as propanolol, pindolol, labetalol, betaxolol; anti-arrythmics, such as procainamide, p rajmaline, disopyramide; antihypertensives, such as clonidine hydrochloride, guanabenz acetate, methyldopa; diuretics, such as ammonium chloride, mannitol, urea, hydrochlorothiazide, bumetanide; vasodilators, such as (general) diazoxide, minoxidil, pinacidil; (Coronary) amotriphene, bendazol, benfurodil hemisuccinate; (Peripheral) bamethan, bencyclane, betahistine; (Cerebral) bencyclane, cinnarizine, citicoline; central nervous system (CNS) stimulants cough and cold preparations, such as dextromethorphan hydrobromide; decongestants, such as pseudoephedrine hydrochloride, diphenhydramine hydrochloride; chlorpheniramine maleate; hormones, such as estradiol, corticosteroids, hydrocortisone; testosterone, progesterone; immunosuppressives, such as cyclosporin, mizoribine, brequinar sodium; parasympatholytics, such as atropine sulfate, belladonna, cyclopentolate hydrochloride; parasympathomimetics, such as pyridostigmine, physostigmine, scopolamine; sedatives, such as buspirone hydrochloride, chloral hydrate, disulfiram; tranquilizers, such as chloropromazine, promazine, fluphenzaine.

In some aspects, the drug may be oxybutynin, buspirone, fentanyl, testosterone, progestin, estradiol, propentofylline, or a mixture thereof. It should be appreciated that one or more of these and other drugs described herein exist in many pharmaceutically acceptable salts. Examples of such salts include those generated by using inorganic agents (i.e., inorganic cations such as sodium, potassium, calcium, etc., and inorganic anions such as chloride, bromide, etc.,) and organic agents (i.e., organic cations such as piperazinyl, triazinyl, etc., and organic anions such as citrates, tartarates, tosylates, etc). In addition, these drugs are also present as polymorphs and/or isomers. Examples of polymorphs include monohydrates, dihydrates, hemihydrates, etc., as well those high-melting and low-melting polymorphs. These polymorphs can be characterized using X-ray crystallographic techniques or other well-known techniques in the art. Examples of isomers include geometric and optical isomers. Further, the pharmaceutical art has recognized that such salts, isomers, and polymorphs, as well as prodrugs, analogs, and metabolites for these drugs can be therapeutically effective as well and can be substituted with ease.

Examples of useful testosterone and related compounds include without limitation: testosterone, methyltestosterone, androstenedione, adrenosterone, dehydroepiandrosterone, oxymetholone, fluoxymesterone, methandrostenolone, testosterone, methyltestosterone, androstenedione, adrenosterone, dehydroepiandrosterone, oxymetholone, fluoxymesterone, methandrostenolone, testolactone, pregnenolone, 17α-methylnortestosterone, norethandrolone, dihydrotestosterone, danazol, oxymetholone, androsterone, nandrolone, stanozolol, ethylestrenol, oxandrolone, bolasterone and mesterolone, testosterone propionate, testosterone cypionate, testosterone phenylacetate, testosterone enanthate, testosterone acetate, testosterone buciclate, testosterone heptanoate, testosterone decanoate, testosterone caprate, testosterone isocaprate, and combinations thereof.

Examples of useful estradiol and related compounds include without limitation: 17β-estradiol, 17α-estradiol, conjugated equine estrogen, esterified estrogen, micronized estradiol, sodium estrogen sulfate, ethinyl estradiol, estrone, tibolone, selective estrogen receptor modulator (SERM), phytoestrogen, and mixtures thereof. Examples of useful progestin and related compounds include without limitation: progesterone, medroxy-progesterone acetate, norethindrone, and norethindrone acetate.

Examples of useful oxybutynin compounds include without limitation: N-desethyloxybutynin, (R)-oxybutynin, (S)-oxybutynin, (R)-N-desethyloxybutynin, and (S)-N-desethyloxybutynin. Particularly, it has been noted that the oxybutynin metabolite, N-desethyloxybutynin, as well as it (R)- and (S)-optical isomers exert an anticholinergic action that is equal to or greater than oxybutynin, and can be readily delivered for such a purpose. See, U.S. Pat. Nos. 5,411,740, 5,500,222, 5,532,278, 5,677,346, 5,686,097, 5,736,577, 5,747,065, 5,750,137, and 5,900,250, which are incorporated by reference in their entirety.

Transdermal delivery of oxybutynin using triacetin as a penetration enhancer has been described by U.S. Pat. No. 5,834,010, and 5,601,839, which are incorporated herein by reference. It is appreciated that transdermal penetration of oxybutynin can be enhanced further by using a quaternary ammonium salt as described by the present invention, and triacetin as a co-enhancer. Oxybutynin can be administered in low concentrations, such that the serum concentrations of one or more of its metabolites can be significantly lowered with the beneficial effect of reduced adverse drug reactions, such as anticholinergic effects (including dry mouth, constipation, blurred vision, etc.). For example, such compositions may comprise an amount of oxybutynin, such that when administered to a subject a plasma area under the curve (AUC) ratio of oxybutynin to an oxybutynin metabolite is from about 0.5:1 to about 5:1. Such oxybutynin compositions have been described in co-pending application Ser. No. 10/098,752, filed on Mar. 15, 2002, which is incorporated herein by reference.

Examples of propentofylline compositions, which can be used in connection with the present invention are described in U.S. Pat. No. 5,762,953, which is incorporated herein by reference. It is appreciated that the transdermal penetration of such compositions may be further enhanced using the quaternary ammonium salt compounds of the present invention.

It is appreciated that any combination of any of the above drugs (that is one or more of any of the above drugs) may be used in this invention. The present invention also contemplates the use of such salts, isomers, polymorphs, prodrugs, analogs, and metabolites, including substances not specifically recited above.

It is appreciated that the above categories of drugs are not rigidly described and that one drug may be described accurately in more than one category or sub-category. For example, insulin may be described as a hormone, as an anti-diabetic agent and also as a macromolecule.

In one aspect, the drug selected for inclusion in the transdermal patch may be fentanyl. As noted above, the amount of fentanyl included may be determined by a number of factors, including the type of polymeric material used for each of the storage and delivery layers, the size or thickness of each, etc. However, in one aspect, the amount of fentanyl in the storage layer may be from about 2% w/w to about 12% w/w of the storage layer, and the amount of fentanyl in the delivery layer may be from about 0.5% w/w to about 1.2% w/w of the delivery layer. In another aspect, the amount of fentanyl in the storage layer may be about 8% w/w of the storage layer, and the amount of fentanyl in the delivery layer may be about 0.8% w/w of the delivery layer.

In use, the transdermal patch of the present invention generally includes a backing member adhered to the storage layer, and a release liner adhered to the delivery layer. The backing member defines the side of the matrix patch that faces the environment, (i.e., distal to the skin or mucosa), and the release liner is adhered to the proximal side of the delivery layer, and must be removed before patch application to the skin. The backing layer functions to protect the storage layer, and to provide an impenetrable layer that prevents loss of delivery substance to the environment. Thus, the material chosen for the backing should be compatible with the polymeric material of the storage layer, drug, and any other agents contained in the layer, and should be minimally permeable to any components of the patch.

Advantageously, the backing can be opaque to protect components of the transdermal patch from degradation caused by exposure to ultraviolet light. Further, the backing member should be capable of binding to and supporting the polymeric material of the storage layer, yet should be pliable to accommodate the movements of a person using the matrix patch.

Suitable materials for the backing member include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene. A thickness of about 0.0005 to about 0.01 inch is preferred. The release liner can be made of the same materials as the backing, or other suitable films coated with an appropriate release surface.

The transdermal patch can further comprise various additives in addition to the polymer layer, delivery substances, and permeation enhancer that are the fundamental components of the adhesive matrix patch formulation. These additives are generally those pharmaceutically acceptable ingredients that are known in the art of transdermal substance delivery and, more particularly, in the art of transdermal substance delivery. However, such additive ingredients must not materially alter the basic and novel characteristics of the matrix patch. For example, suitable diluents can include mineral oil, low molecular weight polymers, plasticizers, and the like. Many transdermal delivery substance formulations have a tendency to irritate the skin after prolonged exposure thereto, thus addition of a skin irritation reducing agent aids may be desirable. One example of a skin irritation reducing agent is glycerin, the use of which is disclosed in U.S. Pat. No. 4,855,294, which is incorporated herein by reference.

### EXAMPLES

The following examples are provided to promote a more clear understanding of certain embodiments of the present invention, and are in no way meant as a limitation thereon.

### Example 1

Transdermal multi-layer adhesive matrix patches of 10 cm² in size were prepared to have a PVP-acrylate storage layer containing either 4% w/w, 6% w/w, or 8% w/w fentanyl, a rate controlling layer of 4.5% w/w EVA, and a silicone adhesive delivery layer having a fentanyl concentration of 0.8% w/w.

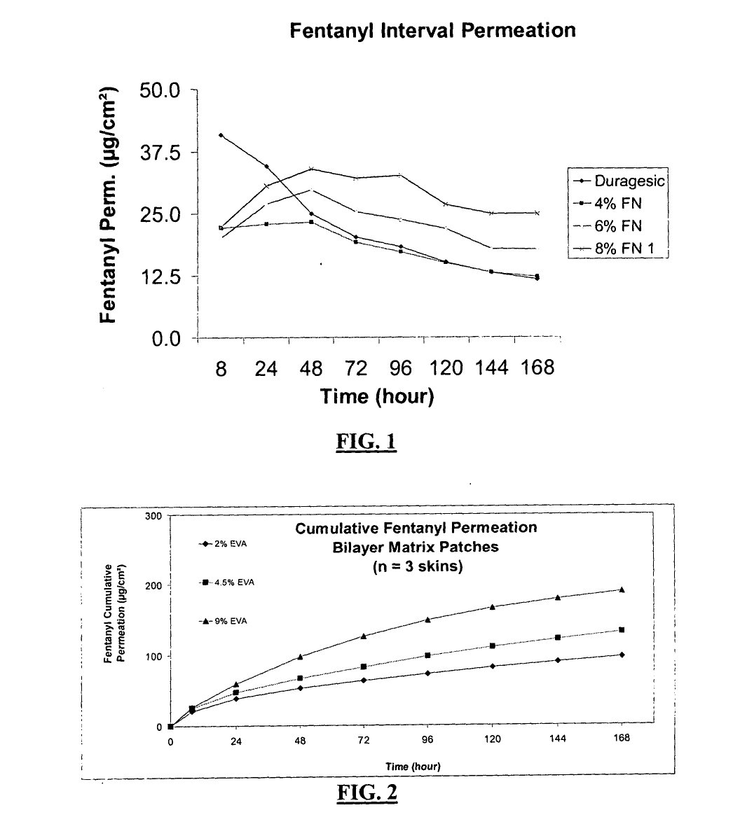

The patches were utilized in a flux study and compared to a 20.4 cm² DURAGESIC™ patch. The mean flux data for the experiment are contained in Table 1 below, and are graphically represented in FIG. 1.

TABLE 1
Comparison of Multilayer Patch Flux to Duragesic ™

| | Mean Data | | | |
|---|---|---|---|---|
| | 8% FN¹ | 4% FN | Duragesic² | 6% FN |
| TIME (hour) | 3 skin donors | 9 skin donors | 3 skin donors | 13 skin |
| 8 | 22 | 22 | 41 | 20 |
| 24 | 31 | 23 | 35 | 27 |
| 48 | 34 | 23 | 25 | 30 |
| 72 | 32 | 19 | 20 | 25 |
| 96 | 33 | 17 | 18 | 24 |
| 120 | 27 | 15 | 15 | 22 |
| 144 | 25 | 13 | 13 | 18 |
| 168 | 25 | 12 | 12 | 18 |

¹Seven day flux data shown only.
²Based on 10 cm² active area.

As be seen, in Table 1, and further in FIG. 1, the drug delivered by the Duragesic™ system started initially higher than the systems of the present invention and has continually diminished with each measured time point from the initial measurement. In fact, the final time point shows that the Duragesic™ patch delivered nearly only one quarter of the amount delivered during the initial time point. By contrast, the multilayer systems of the present invention have a final time point flux value that is no less than one half of the starting value. Further, the 6% patch has a final flux value that is 90% of the initial flux, and the 8% patch has a final flux that is actually greater than the initial flux, suggesting that release of the fentanyl in a therapeutic amount could be sustained well beyond the 168 hour (7 day) time point.

### Example 2

Transdermal multilayer patches of 10 cm² in size were made as in Example 1, except each patch had an identical amount of fentanyl in the storage layer of 4% w/w. The only variation between the patches was the amount of EVA in the rate controlling member. The amount of EVA was varied at 2% w/w, 4.5% w/w, and 9% w/w respectively. The flux results for the patches are shown in Table 2 below, and in FIG 2.

TABLE 2
Flux Study of Patches with Varied EVA Content

| | 2% EVA | 4.5% EVA | 9% EVA |
|---|---|---|---|
| | 2% EVA top: 12 | 4.5% EVA. top: 12 | 9% EVA top: 12 |
| | mil 87-2888 | mil 87-2888 | mil 87-2888 |
| TIME | bottom: 7 mil | bottom: 7 mil | bottom: 7 mil |
| (hour) | 7-4302. | 7-4302. | 7-4302. |
| 0 | 0 | 0 | 0 |
| 8 | 21 | 26 | 27 |
| 24 | 39 | 47 | 59 |
| 48 | 53 | 67 | 97 |
| 72 | 63 | 82 | 126 |
| 96 | 73 | 98 | 149 |
| 120 | 82 | 110 | 166 |
| 144 | 89 | 121 | 179 |
| 168 | 96 | 131 | 189 |

As can be seen in Table 2 and FIG. 2, the higher amounts of EVA improved the flux rates for the patches over time.

It is to be understood that the above-described devices and methods are only illustrative of preferred embodiments of the present invention. Numerous modifications and alternative arrangements may be devised by those skilled in the art without departing from the spirit and scope of the present invention and the appended claims are intended to cover such modifications and arrangements.

Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A transdermal patch for administering a drug to a subject comprising:
a storage layer having an amount of the drug contained substantially therein, and having a proximal side and a distal side;
a delivery layer having an amount of the drug contained substantially therein, and having a proximal side and a distal side;
a backing member adhered to the distal side of the storage layer;
a rate controlling member adhered between the proximal side of the storage layer and the distal side of the delivery layer; and
a peelable release liner adhered to the proximal side of the delivery layer, wherein the release liner is removed prior to application of the patch to the skin of the subject.

2. The transdermal patch of claim 1, wherein the storage layer comprises a polymeric material selected from the group consisting of: acrylate polymers, vinyl polymers, silicone polymers, polyisobutylene polymers, copolymers thereof, and mixtures thereof.

3. The transdermal patch of claim 2, wherein the polymeric material is a polyvinyl pyrrolidone polymer.

4. The transdermal patch of claim 3, wherein the polyvinyl pyrrolidone polymer is a polyvinyl pyrrolidone acrylate.

5. The transdermal patch of claim 1, wherein the delivery layer comprises a polymeric adhesive material selected from the group consisting of: acrylate polymers, vinyl polymers, silicone polymers, polyisobutylene polymers, copolymers thereof, and mixtures thereof.

6. The transdermal patch of claim 5, wherein the polymeric adhesive material is a silicone polymer.

7. The transdermal patch of claim 1, wherein the rate controlling member includes a material selected from the group consisting of: microporous membranes, dense materials, and polymeric materials.

8. The transdermal patch of claim 7, wherein the rate controlling member is a layer that includes ethylene vinyl acetate.

9. The transdermal patch of claim 1, wherein the amount of drug initially present in the storage layer is from about 4 to about 15 times the amount of drug in the delivery layer.

10. Transdermal patch of claim 9, wherein the amount of drug initially present in the storage layer is about 5 times the amount of drug in the delivery layer

11. The transdermal patch of claim 1, wherein the drug is fentanyl.

12. The transdermal patch of claim 11, wherein the amount of fentanyl in the storage layer is from about 2% w/w to about 12% w/w of the storage layer, and the amount of fentanyl in the delivery layer it is from about 0.5% w/w to about 1.2% w/w of the delivery layer.

13. The transdermal patch of claim 12, wherein the amount of fentanyl in the storage layer is about 8% w/w of the storage layer, and the amount of fentanyl in the delivery layer is about 0.8% w/w of the delivery layer.

14. A method of providing extended transdermal delivery of a drug to a subject, comprising:
administering a transdermal patch as recited in claim 1 to the skin of the subject.
